(19) United States
(12) Patent Application Publication
(10) Pub. No.: US 2026/0097389 A1
(43) Pub. Date: Apr. 9, 2026

(54) Cellulose-based absorbent material and process for its production

(71) Applicant: Teknologian tutkimuskeskus VTT Oy, Espoo (FI)

(72) Inventors: Ali Harlin, Espoo (FI); Lauri Hauru, Espoo (FI); Harri Setälä, Espoo (FI)

(21) Appl. No.: 19/112,665

(22) PCT Filed: Sep. 29, 2023
(86) PCT No.: PCT/FI2023/050556

§ 371 Date: Mar. 18, 2025

(30) Foreign Application Priority Data
Sep. 30, 2022 (FI) 20225871

(51) Int. Cl. B01J 20/24; A61L 15/28; B01J 20/28; B01J 20/30; C08B 11/02; C08B 11/20; C08B 15/06

(52) U.S. Cl. CPC B01J 20/24; A61L 15/28; B01J 20/28004; B01J 20/28023; B01J 20/28047; B01J 20/3007; B01J 20/3085; C08B 11/02; C08B 11/20; C08B 15/06

(57) ABSTRACT

The present invention relates to a process for producing a cellulose-based biodegradable superabsorbent material, suitable for use in applications such as wet wipes, disposable packaging, hygiene products, medical devices and a vast variety of other technical applications requiring a combination of high absorption and water hold-up. The invention also relates to the thus obtained material.

### BACKGROUND OF THE INVENTION

### Field of the Invention

The present invention relates to a process for producing a cellulose-based biodegradable superabsorbent material that is suitable for use in applications requiring a combination of high absorption and water hold-up, such as personal hygiene products, medical devices and a vast variety of other technical applications. The invention also relates to the thus obtained material.

### Description of Related Art

A superabsorbent polymer (SAP) is a polymer that can absorb large amounts of aqueous liquids. This absorption makes the SAP swell, whereby the absorbency of the SAP is often characterized using its swelling capacity.

The SAPs currently on the market are mainly synthetic and non-biodegradable, commonly based on polyacrylic acid, blended with sodium hydroxide, thus forming a sodium polyacrylate. Due to the synthetic non-biodegradable character of the product, there is a need for replacing these common SAPs with biodegradable and renewable alternatives.

Cationized starch has been used in absorbents in the past, since the developments of such products started in the early 1960s, but these starch-based absorbents lack the cohesion that is necessary for such products.

Cellulose-based absorbents have also been used in the past, even before synthetic absorbents were developed, some of the oldest alternatives consisting of tissue paper, cotton or fluff pulp.

Also more recent developments have been made to cellulose absorbents, e.g. as described by Hubbe et al. (2013). Typically, these more recent cellulose absorbents have, however, used functionalized cellulose derivatives, thus causing a smaller level of absorption as compared to synthetic SAPs, with a free swelling capacity (FSC) of 5-10 g/g and a low absorption under load (AUL), or cellulose grafted with acryl groups or the like and crosslinked, thus compromising the biodegradability and compostability of the product. WO 2012127119 A2 describes such a material with a polysaccharide backbone that has been functionalized and cross-linked.

At least partly due to these disadvantages of known cellulose-based absorbents, synthetic polyacrylate-based SAPs are still more common than cellulose-based alternatives. They have high absorbency and AUL, but are non-biodegradable, and cannot be recycled.

Therefore, there is a strong interest towards further development of biodegradable and recyclable absorbent materials that have a high absorption capacity in addition to being biodegradable.

### SUMMARY OF THE INVENTION

The invention is defined by the features of the independent claims. Some specific embodiments are defined in the dependent claims.

According to a first aspect of the present invention, there is provided a process for preparing a cellulose-based absorbent material requiring no crosslinking, since the crosslinking can be replaced by a natural cohesion between the cellulose moieties.

According to a second aspect of the invention, there is provided a process for preparing a cellulose-based absorbent material, by utilizing a hydroxypropylation step to increase the accessibility of the cellulose.

According to a third aspect of the invention, there is provided a process for preparing a cellulose-based absorbent material, where the high absorbency is achieved by a cationization step.

According to a fourth aspect of the invention, this functionalized material is prepared with a low degree of substitution (DS), to avoid compromising the biodegradability of the material.

According to a further aspect of the invention, there is provided a cellulose-based absorbent material suitable for use in wet wipes or other similar hygiene products required to absorb high amounts of liquid, disposable packaging, personal hygiene products, such as diapers or feminine hygiene products, in medical devices or in technical applications, such as agricultural applications, to provide high absorption and water retention.

The invention thus relates to a process for producing a biodegradable absorbent material, including the functionalization of a polymeric raw material selected from cellulose, without crosslinking, by combining a low-DS hydroxypropylation step and a low-DS cationization step.

Several advantages are achieved using said invention. Among others, a renewable feedstock can be used, and simple chemistry. Since the combination of the low-DS hydroxypropylation step and the low-DS cationization step carried out on the cellulose raw material results in a product still having a low DS, a biodegradable and recyclable product is also obtained.

Further, these steps result in a high absorbency material having a free swelling capacity (FSC) that is several times higher than for known cellulose-based crosslinked absorbents (typically >35 g/g). Said absorbency is competitive with commercial synthetic SAPs.

The lack of explicit crosslinking and a low DS further enables an efficient utilization of the bulk of the cellulose, while not compromising biodegradability.

It would be possible to prepare an absorbent also without the separate hydroxypropylation step, i.e. by directly cationizing the cellulose raw material. However, in such a case, achieving the desired degree of substitution (DS) would require a larger amount of cationizing reagent, which in case of the commonly used glycidyl trimethyl ammonium chloride (GTAC) is both expensive and harmful. The separate hydroxypropylation activates the cellulose by opening up its structure, thus helping the cationization reagent to reach the sites of reaction on the cellulose, and increasing the absorbency of the product.

Likewise, it would be possible to prepare an absorbent also without the separate cationizing step, i.e. by directly fluidizing the hydroxypropylated material. However, in such a case, preparing a properly fluidized product would require increasing the DS value in the hydroxypropylation, whereby it would not be possible to obtain a low-DS super-absorbent.

The separate fluidization step is, in turn, needed specifically for the low-DS product, as a low-DS material has been found not to form a dispersed gel-like material without said step.

### EMBODIMENTS OF THE INVENTION

### Definitions

In the present context, the “absorbency” of a material is defined as its free swelling capacity, which for both conventional synthetic non-biodegradable SAPs and for the cellulose-based SAPs of the present invention is high, typically >30 g/g.
The cellulose raw material of the process is turned into a chemically functionalized cellulose.
The degree of substitution (DS) of the cellulose material after its functionalization is described in terms of “low DS” when the DS value for the absorbent, or the intermediate products obtained during its preparation, is <2. In the present materials, the DS values are preferably even lower, with values of <0.5. With the low DS, the material still resembles native cellulose enough to be biodegradable.

The present invention relates to a process for producing a biodegradable absorbent material, including the functionalization of a polymeric raw material selected from cellulose materials.

The process is carried out without crosslinking, by combining a low-DS hydroxypropylation step and a low-DS cationization step, and is characterized by
providing a polymeric raw material formed of cellulose,
hydroxypropylating and cationizing the cellulose raw material to form a chemically functionalized material having a degree of substitution (DS) of <0.5,
fluidizing the chemically functionalized material to turn it into a fibrillated gel-like material, and
drying the fibrillated gel-like material to obtain a cellulose-based biodegradable absorbent product.

The cellulose raw material is preferably dissolving pulp. Particularly, pre-treated and pre-functionalized celluloses are avoided, since they may result in disadvantageous consistencies or deficient biodegradability.

The hydroxypropylation step is intended to increase the accessibility of the cellulose, and is typically carried out using propylene oxide as the hydroxypropylation reagent. The temperature during the reaction is preferably between 25-70° C., and is maintained for a suitable duration to achieve the desired reaction, such as for a duration of 3-10 h, typically using an excess of hydroxypropylating reagent, such as >3 molar equivalents.

Typically, the reaction taking place during the hydroxypropylation step is the reaction of the following Scheme 1.

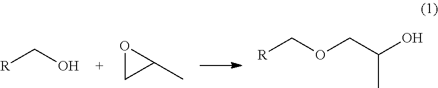

In an embodiment of the invention, the hydroxypropylation is carried out to a degree of substitution (DS) of ≤0.5, preferably 0.2-0.5, or most suitably 0.3-0.4.

The cationization, in turn, provides the cellulose with the functional groups needed for the high absorbancy. This cationization step is typically carried out using glycidyl trimethyl ammonium chloride (GTAC) as the cationization reagent. The temperature during the reaction is preferably between 40-80° C., and is maintained for a suitable duration to achieve the desired reaction, such as for a duration of 6-20 h, typically using an excess of cationization reagent, such as >4 molar equivalents.

Typically, the reaction taking place during the cationization step is the reaction of the following Scheme 2.

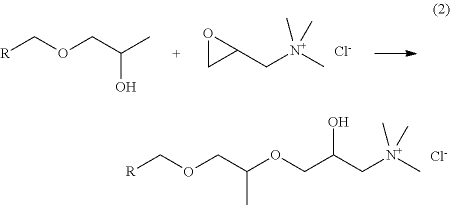

In an embodiment of the invention, the cationization is carried out to a degree of substitution (DS) of ≤0.4, preferably ≤0.2, or most suitably about 0.15.

In a preferred embodiment of the invention, the cationization is carried out directly on the hydroxypropylated material, without isolating the hydroxypropylated material. This is possible, since the hydroxypropylation reaction and the cationization reaction are carried out in similar environments. Further, each isolation step typically reduces the yield of product, whereby it is advantageous to omit them when possible.

Achieving the desired degree of substitution is important for the present invention in order to obtain a natural and biodegradable product, and both the hydroxypropylation and the cationization steps are typically optimized to facilitate achieving this goal. However, the amounts of hydroxypropylating reagent and cationization reagent added to the cellulose can also be adjusted, in order to control the relative contribution of cellulose to the mass of the final product. In terms of dry matter, the final functionalized absorbent product should be comprised of 84-88 w-% cellulose equivalent, preferably about 86 w-%. The remainder of the mass is contributed by the hydroxypropyl moieties grafted onto the cellulose.

The following step of the process, after the hydroxypropylation and cationization, is the fluidization step. This step is essential to obtain a gel-form material. Preferably, the fluidization is carried out in a controlled manner, more preferably with the material in an aqueous suspension at a solids content of 0.3-0.75 w-%, such as about 0.5 w-%. Most suitably, microfluidization is used, typically using more than one pass through a microfluidizer, preferably 2-3 passes, to achieve a fibre length of <30 μm.

Once a suitable gel-like consistency has been achieved for the fluidized material, the material can be dried. The drying is typically carried out in a controlled manner, preferably by freeze-drying, to avoid collapsing the achieved structure.

As a conclusion of the above, the process of the invention typically includes four process steps:
1) slight hydroxypropylation
2) cationization without isolation of the hydroxypropylated material
3) fluidization, and
4) drying the obtained gel.

The above described process results in a biodegradable cellulose-based absorbent material. Thus, the present invention also relates to said material, prepared using the above process.

A preferred degree of substitution (DS) for this material is <0.5, since a sufficiently low DS value will ensure that the material is still biodegradable.

In addition to having the biodegradability, the material of the invention needs to provide a sufficient absorption. Thus, the biodegradable cellulose-based absorbent material of the invention preferably has a free swelling capacity (FSC) of >15 g/g, more preferably >30 g/g, and most suitably >35 g/g. Further, the material preferably has an absorption under load (AUL) of >8 g/g, more preferably 10 g/g and most preferably >12 g/g in saline (0.9% NaCl) or in synthetic urine (3.0% urea, 1.0% sodium chloride, 0.4% potassium chloride, 0.3% sodium sulfate).

The biodegradable cellulose-based absorbent material described above is suitable for use particularly in wet wipes or other similar hygiene products that require absorption of large amounts of aqueous solutions, disposable packaging, personal hygiene products, such as diapers or feminine hygiene products, in medical devices or in technical applications, such as agricultural applications, to provide high absorption and water retention.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. In addition, various embodiments and examples of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In this description, numerous specific details are provided to provide a thorough understanding of embodiments of the invention. One skilled in the relevant art will recognize, however, that the invention can be practiced without one or more of the specific details.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The following non-limiting examples are intended merely to illustrate the advantages obtained with the embodiments of the present invention.

### EXAMPLES

### Example 1—Preparation of Cellulose-Based Superabsorbent Polymer

Dissolving pulp, 430 ml/g, was obtained from Domsjö and used as cellulose raw material. Samples of the material was hydroxypropylated at 25° C. for 4 h using an excess of propylene oxide (4.1 molar equivalents) to a DS of ≤0.5, and directly cationized at 45° C. for 16 h using an excess of GTAC (5.0 molar equivalents), without isolation of the hydroxypropylated material, to a DS of about 0.15. The obtained cationized material was fluidized by passing it through a fluidizer (M-110EH, Microfluidics Ind.) using three passes (1000 bar, 1800 bar and 1800 bar). The end-point of the fluidizing step was determined by microscopy and selected as the point when visible fibers had mostly disappeared. The thus obtained material in gel-form was dried by freeze-drying.

Control samples were prepared by either omitting the functionalization (hydroxypropylation and cationization) or the fluidizing, or both.

The free swelling capacity in water at 5 min (FSC) was 41-58 g/g for the SAP, measured using an open cage. FSC in synthetic urine was 22 g/g and absorption under load (AUL, 2 kPa/0.3 psi static pressure) in synthetic urine was 12 g/g. FSC was also measured for the controls. FSC was 14 g/g for unfunctionalized-fluidized control. The functionalized-unfluidized control material disintegrated and could not be retained by the cage, but its swelling capacity could measured in a teabag, where it was 11 g/g. AUL was measured with a method adapted from Bachra et al.

### INDUSTRIAL APPLICABILITY

The present material can be used in a vast variety of applications requiring high absorption and water retention, and generally for replacement of conventional non-biodegradable synthetic absorbents.

In particular, the present material is useful in personal hygiene products, such as wet wipes, disposable packaging, and hygiene products, in medical devices or in agricultural applications.

### CITATIONS

Bachra, Y., Grouli, A., Damiri, F., Bennemara, A., Berrada, M. A new approach for assessing the absorption of disposable baby diapers and superabsorbent polymers: A comparative study. Results in Materials 8 (2020) 100156, doi.org/10.1016/j.rinma.2020.100156
Hubbe et al. (2013) “Review of cellulosic absorbents.” BioResources 8(4) 6556-6629 WO 2012127119 A2

1. A process for producing a biodegradable absorbent material, the process comprising:
providing a cellulose raw material,
hydroxypropylating and cationizing the cellulose raw material to form a chemically functionalized material having a degree of substitution (DS) of <0.5,
fluidizing the chemically functionalized material to form a fibrillated gel-like material, and
drying the fibrillated gel-like material to obtain a cellulose-based biodegradable absorbent product.

2. The process of claim 1, wherein the cellulose raw material is dissolving pulp.

3. The process of claim 1, wherein the hydroxypropylation is carried out to a degree of substitution (DS) of <0.5.

4. The process of claim 1, wherein the hydroxypropylation is carried out using propylene oxide as a hydroxypropylating reagent at a temperature of 25-70° C. for 3-10 h using an excess of the hydroxypropylating reagent.

5. The process of claim 1, wherein the cationization is carried out to a degree of substitution (DS) of ≤0.2.

6. The process of claim 1, wherein the cationization is carried out using glycidyl trimethyl ammonium chloride (GTAC) as a cationization reagent a temperature of 40-80° C. for 6-20 h using an excess of the cationization reagent.

7. The process of claim 1, wherein the cationization is carried out directly on the hydroxypropylated material, without isolating the hydroxypropylated material.

8. The process of claim 1, wherein an amount of a hydroxypropylating reagent and a cationization reagent are added to the cellulose, and wherein the amount of the hydroxypropylating reagent and the cationization reagent is adjusted to give a cellulose equivalent of 84-88 w-% of the dry matter, in the final absorbent product.

9. The process of claim 1, wherein the fluidizing is carried out in a controlled manner with the material in an aqueous suspension at a solids content of 0.3-0.75 w-%.

10. The process of claim 1, wherein the drying is carried out by freeze-drying, to avoid collapsing the cellulose-based biodegradable absorbent product.

11. A biodegradable cellulose-based absorbent material prepared using the process of claim 1.

12. The biodegradable cellulose-based absorbent material of claim 11, having a degree of substitution (DS) of <0.5.

13. The biodegradable cellulose-based absorbent material of claim 11, having a free swelling capacity (FSC) of >15 g/g.

14. The biodegradable cellulose-based absorbent material of claim 11, wherein the biodegradable cellulose-based absorbent material is, or is incorporated into, one or more of disposable packaging, personal hygiene products, diapers, feminine hygiene products, medical devices or agricultural products, to provide absorption and water retention.

15. The process of claim 9, wherein fluidizing is carried out by microfluidization through a microfluidizer to achieve a fibre length of <30 μm.
